(19) United States
(12) Patent Application Publication
(10) Pub. No.: US 2010/0030095 A1
(43) Pub. Date: Feb. 4, 2010

(54) Pulse Acoustic Analysis System for the Diagnostic of Cardiovascular Disease

(75) Inventor: Fan-Ming Yu, Tainan City (TW)

Correspondence Address: WPAT, PC;INTELLECTUAL PROPERTY ATTORNEYS, 2030 MAIN STREET, SUITE 1300, IRVINE, CA, 92614, US

(73) Assignee: Sustineo Biotechnology, Hsinchu City (TW)

(21) Appl. No.: 12/182,510

(22) Filed: Jul. 30, 2008

(51) Int. Cl. A61B 5/024

(52) U.S. Cl. 600/526; 600/528

(57) ABSTRACT

This invention provides a pulse acoustic analysis system for detecting disease. The present invention further provides a method for detecting a dynamic pressure change in a blood dynamic system in human.

### FIELD OF THE INVENTION

The present invention relates to a method for detecting a dynamic pressure change in a blood dynamic system in a subject.

This invention further relates to a pulse acoustic analysis system for detecting a cardiovascular disease or condition.

### DESCRIPTION OF PRIOR ART

The use of a radial arterial pulse diagnostic instrument for medication is the state of art in Chinese medical examination technology. However, owing to the improper selection of the sensing element in these instruments, it can only detect the mean radial arterial pressure wave and can not catch the detailed variation of pressure wave signal of the cardiovascular system. The spectrum obtained by Fourier transformation of the measured variation of pressure wave can not offer the complete hemodynamics behavior of the cardiovascular system.

There are some other non-invasive methods for detecting blood volume changes such as Photoplethysmography or the use of pulse wave analysis and a modified Windkessel model.

Photoplethysmography (PPG) is a simple and low-cost optical technique that can be used to detect blood volume changes in the microvascular bed of tissue. It is often used non-invasively to make measurements at the skin surface. Although the origins of the components of the PPG signal are not fully understood, it is generally accepted that they can provide valuable information about the cardiovascular system. The PPG technology has been used in a wide range of commercially available medical devices for measuring oxygen saturation, blood pressure and cardiac output, assessing autonomic function and also detecting peripheral vascular disease (Physiological Measurement, 28(3), R1-R39 (2007)). However, it can only measure the blood volume changes, but can not detect absolute level of blood volume.

Another noninvasive technique, with the use of pulse wave analysis and a modified Windkessel model, has been developed and validated for calculating capacitive and oscillatory systemic arterial compliance. Use of the technique to subjects with hypertension, postmenopausal women with symptomatic coronary artery disease, and appropriate control subjects has confirmed a reduction of oscillatory compliance in the disease states and an increase in capacitive and oscillatory compliances in response to vasodilator drugs. This method should be useful in screening subjects for early evidence of vascular disease and in monitoring the response to therapy. However, the validity and utility of Windkessel-derived variables is further diminished by the absence of between-site correlations and the common occurrence of uninterpretable values in hypertensive subjects.

U.S. Pat. No. 5,913,826 disclosed an apparatus for assessing cardiovascular status of a mammal comprising a system for locally applying a pressure to an artery, capable of restricting blood flow through said artery, a wideband external pulse transducer, having an output, situated to measure acoustic signals proximate to said artery, and a computing device receiving said output for calculating, based on said output, a peripheral vascular impedance value. However, this apparatus occludes arterial blood flow which is not desirable for human health.

### BRIEF DESCRIPTION OF THE DRAWINGS

FIG. 1 shows the pulse acoustic graph of radial artery.

FIG. 2 shows the pulse acoustic analytical chart of radial artery. In the figure, C1 indicates heart meridian; C2 indicates liver meridian; C3 indicates kidney meridian; C4 indicates spleen meridian; C5 indicates lung meridian; C6 indicates stomach meridian; C7 indicates gall meridian; C8 indicates urinary bladder meridian; C9 indicates large intestine meridian; C10 indicates tri-Jiao meridian; C11 indicates small intestine meridian; and C12 indicates pericardium meridian.

FIG. 3 shows the setup of pulse acoustic analysis system. In this figure, 10, 20, 30 and 40 indicate the pulse acoustic sensor, the digital-to-analog converter, the computer, and the oscilloscop respectively.

FIG. 4 shows (A) the pulse acoustic graph, (B) electrocardiograph and (C) arterial blood pressure graph from a patient with heart valve insufficiency. The valve insufficiency could not be detected by either the electrocardiograph or arterial blood pressure analyzer, but could be detected by pulse acoustic analysis system. The plateau in the pulse acoustic graph indicates the heart valve insufficiency.

FIG. 5 shows (A) the pulse acoustic graph, (B) electrocardiograph and (C) arterial blood pressure graph from a patient without heart valve insufficiency.

FIG. 6 shows (A) the original waveform and (B,C) Fourier-transformed wave data, which indicate the blood flow to each organ as depicted in FIG. 2, from the person examined with pulse acoustic analysis system. The difference between (B) and (C) is that the transformed wave data are normalized in (C).

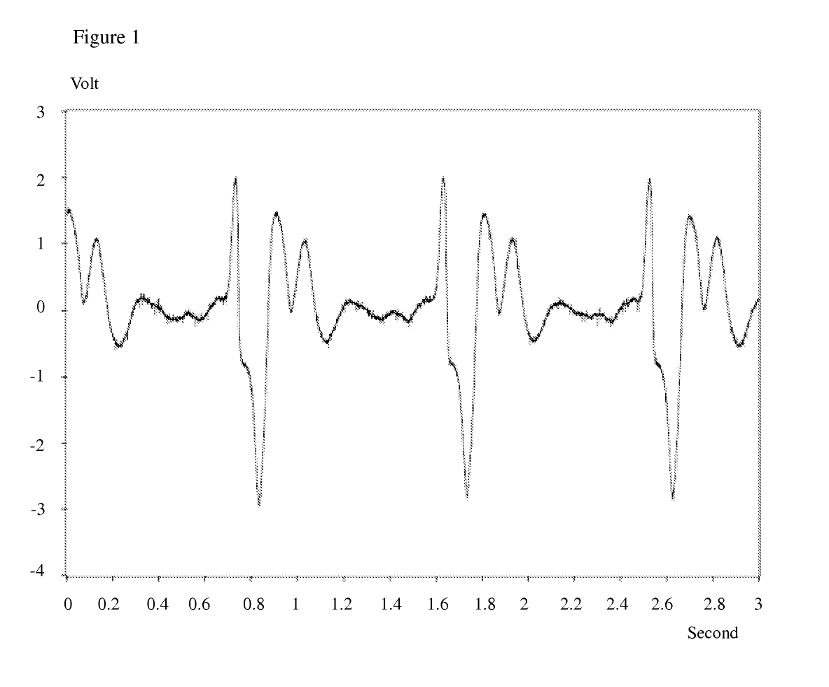

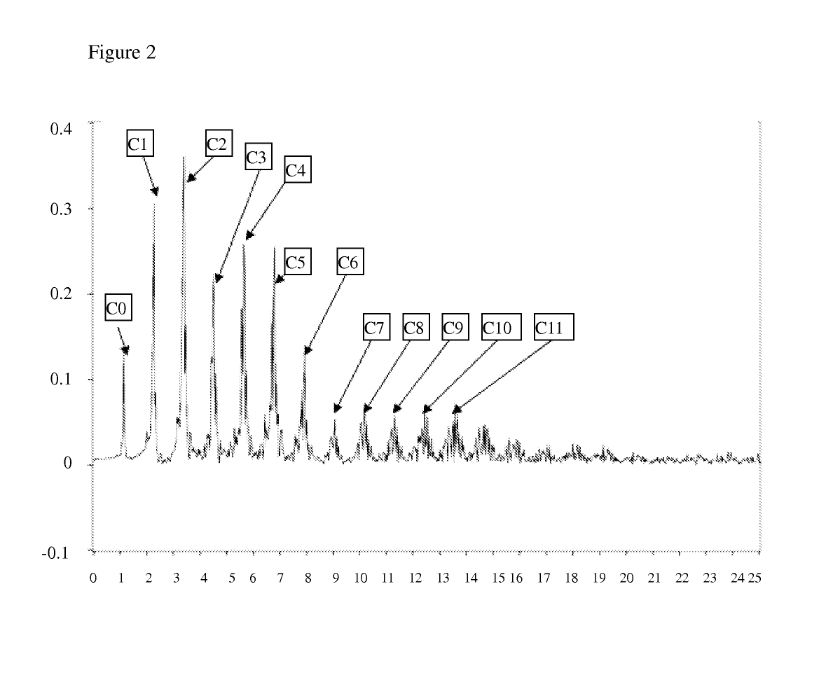

### SUMMARY OF THE INVENTION

The present invention provides a pulse acoustic analysis system for detecting a cardiovascular disease or condition, comprising:
(a) a miniature acoustic sensor;
(b) a tool for fixing the miniature acoustic sensor;
(c) a signal amplifier connected to the miniature acoustic sensor;
(d) a digital-to-analog converter connected to signal amplifier; and
(e) a device, having a data collecting and processing program, which is connected to the digital-to-analog converter.

The present invention further provides a method for detecting a dynamic pressure change in a blood dynamic system in a subject, comprising the following steps:
(a) fixing a pulse acoustic sensor on an artery to detect a pulse acoustic signal;
(b) amplifying the pulse acoustic signal by a signal amplifier;
(c) retrieving the signal by a digital-to-analog converter and storing it in a computer;
(d) conducting a waveform analysis of the signals by a data collecting and processing program to generate a result of amplitude and frequency; and analyzing correlation between the result and the subject's health.

### DETAILED DESCRIPTION OF THE INVENTION

On resolving this problem as stated in the prior art, an audiocardiography system has been developed and tested based on the hemodynamics theory. The sound of blood pressure wave which is originated from heart and being propagating along the radial artery has been measured and examined. This signal contains detailed hemodynamics information in a cardiovascular system. These data are vital for the diagnosis on the cardiac disorders. The digital signal analysis of the obtained data can unveil the detailed hemodynamics behavior. The analysis of signal in-line with electrocardiograph wave and mean arterial pressure wave delivers detail information corresponding to the cardiovascular system. This device can offer an early detection on the cardiac disorders.

The present invention provides pulse acoustic analysis system for diagnosing cardiovascular condition, comprising:
(a) a miniature acoustic sensor;
(b) a tool for fixing the miniature acoustic sensor;
(c) a signal amplifier connected to the miniature acoustic sensor;
(d) a digital-to-analog converter connected to signal amplifier; and
(e) a device, having a data collecting and processing program, which is connected to the digital-to-analog converter.

In a preferred embodiment, the disease is a cardiovascular disease and the system diagnoses a dynamic state of blood output of a visceral organ.

Preferably, the visceral organ is a heart and a blood-vessel-related system.

In a preferred embodiment, the device, having a data collecting and processing program, is a computer, and the data collecting and processing program is Fourier transformation.

In a further preferred embodiment, the tool is a wrist band which fixes the miniature acoustic sensor on a radial artery.

In another preferred embodiment, the system of present invention further comprises an oscilloscope connected to the signal amplifier.
1. The present invention further provides a method for detecting a dynamic pressure change in a blood dynamic system in a subject, comprising the following steps:
(a) fixing a pulse acoustic sensor, which consists of the miniature acoustic sensor and the tool of claim 1, on an artery to detect a pulse acoustic signal;
(b) amplifying the pulse acoustic signal by a signal amplifier;
(c) retrieving the signal by a digital-to-analog converter and storing the retrived signal in a computer;
(d) conducting a waveform analysis of the retrieved signals by a data collecting and processing program to generate a result of amplitude and frequency; and
(e) analyzing correlation between the result and the subject's health.

In a preferred embodiment, the artery is radial artery, carotid artery or dorsalis pedis.

In another preferred embodiment, the analysis comprises original waveform analysis, pulse acoustic waveform analysis and frequency spectrum analysis, and the original waveform and the pulse acoustic waveform are displayed on a screen.

Preferably, the data collecting and processing program is Fourier transformation.

In a preferred embodiment, the original waveform analysis reveals a systole and a diastole of the heart and an on-off switch of cardiac valves.

In another preferred embodiment, the frequency of the pulse acoustic waveform analysis reveals information of organs in the subject and the amplitude of the pulse acoustic waveform analysis reveals blood supply information of organs in the subject.

In one embodiment, the original waveform and the pulse acoustic waveform are output simultaneously, and compared, further with an electrocardiograph and an arterial blood pressure graph, to identify dynamic information of heart and blood vessel and dynamic state of blood output of branching blood vessels.

In another embodiment, the correlation between the result and the subject's health is further classified according to the type of cardiovascular disease.

The example below is non-limiting and is merely representative of various aspects and features of the present invention.

### EXAMPLES

### Example 1

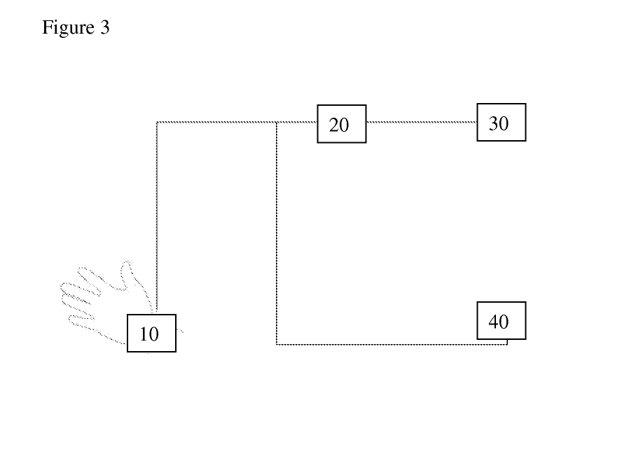

The setup of pulse acoustic analysis system was shown in FIG. 3. The pulse acoustic sensor in the figure detected pulse acoustic signals from radial artery, and the signals were amplified by a signal amplifier and retrieved by digital-to-analog converter and then stored in a computer, and simultaneously, it could be monitored by an oscilloscope; data collecting and processing program were written by NI LABVIEW 8.0 software. This program could show original waveform and pulse acoustic frequency spectrum on the screen separately which could be used to confirm if the experiment was successful.

This example included two parts: one was the development and test of the pulse acoustic analysis system and the other was the analysis and process of the medical data of the pulse acoustic analysis system.

The development and test of the pulse acoustic analysis system:

1. Using components of electronic products in the market to design and test the miniature acoustic sensor.

2. Using the standard acoustic sensor to calibrate the home-made miniature acoustic sensor and identify the frequency and bandwidth of the signals which it detected.

3. Designing and producing a wrist band which can immobilize the miniature acoustic sensor stably and this wrist band can fix the miniature acoustic sensor on the radial artery.

4. Combining the wrist band of miniature acoustic sensor, electricity generator, oscilloscope and digital-to-analog converter and personal computer to become a pulse acoustic analysis system, and then testing the function of the system.

5. Using NI Labvew 8.0 software to develop the high efficiency program of data collection and analysis.

The analysis and process of the medical data of the pulse acoustic analysis system:

1. Outputting simultaneously the signals of the pulse acoustic analysis system, electrocardiogram and blood pressure wave and storing them in the computer.

2. Collecting the data of the signals of the pulse acoustic analysis system, electrocardiogram and blood pressure wave in the instrument room of cardiac catheterization treatment room.

3. Analyzing pulse acoustic waveform and frequency spectrum and obtaining the information of heard, blood vessel and blood dynamics from the characteristics of waveform and frequency spectrum. Analyzing the relationship between the information and disease, and then comparing and classifying them based on different types of cardiac diseases for the diagnosis of cardiac disease patients in the future.

### Example 2

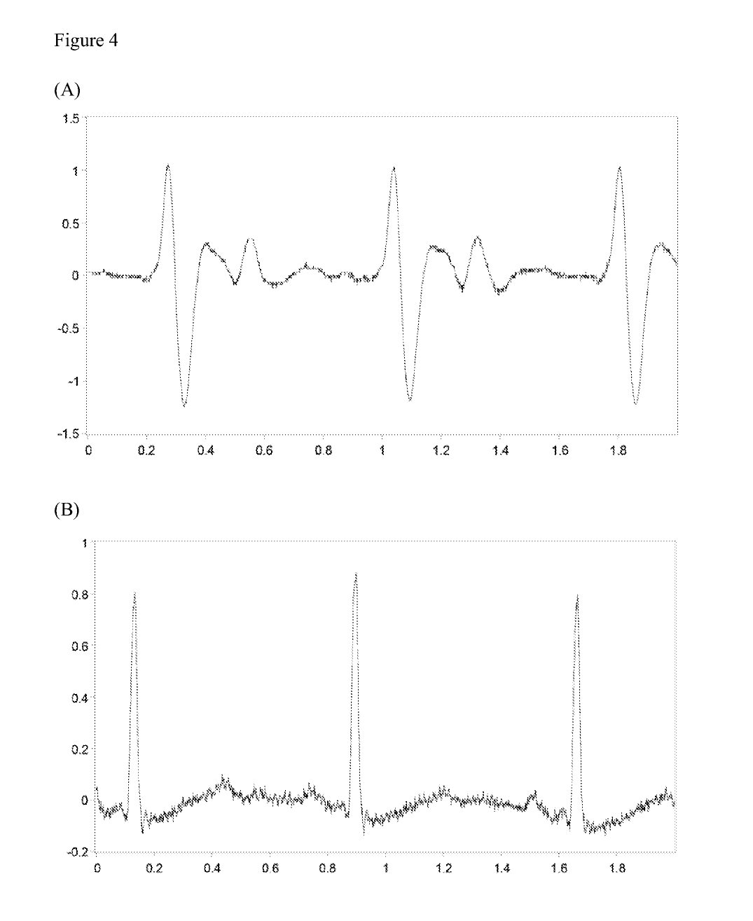

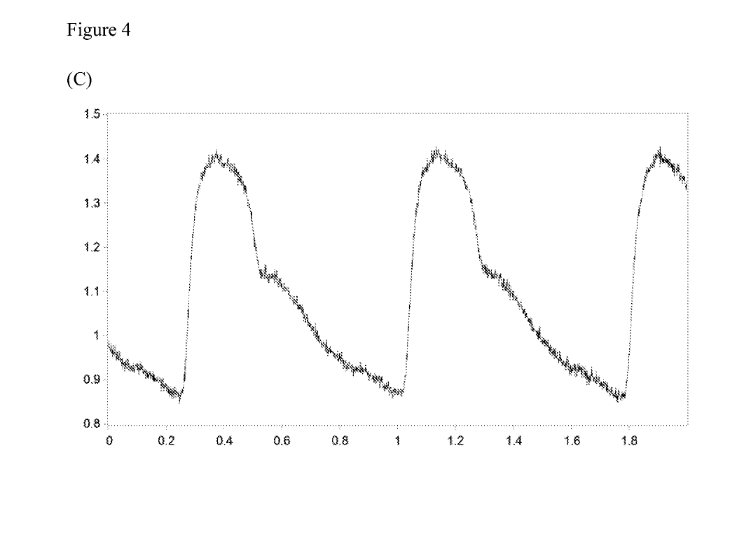

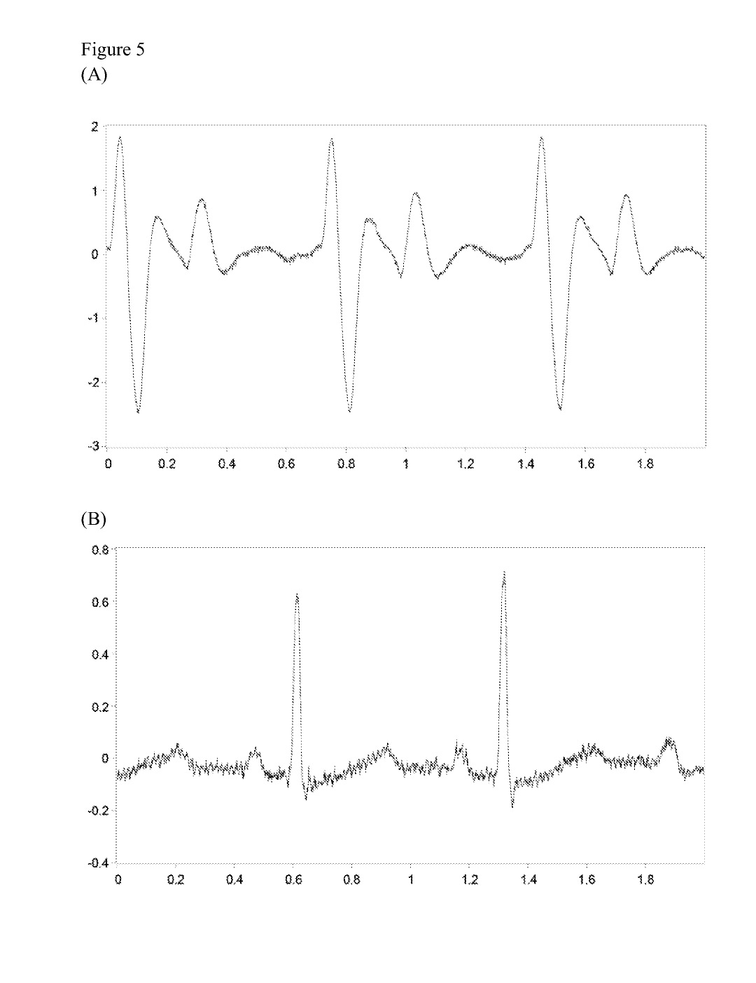

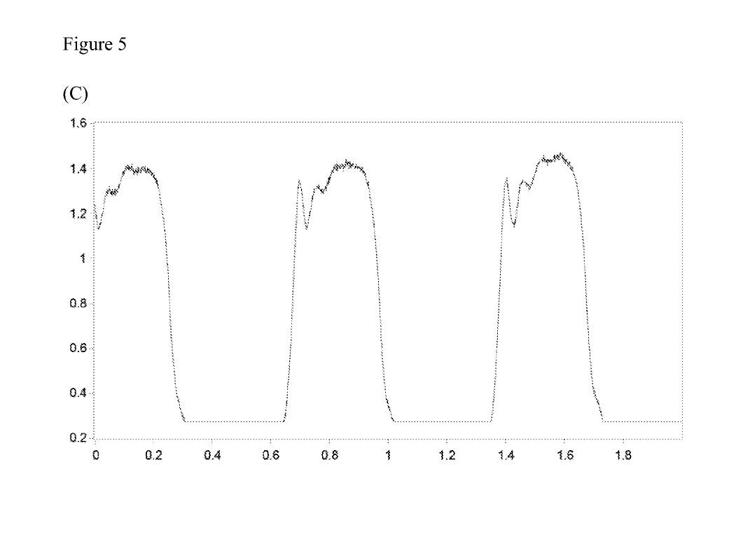

Three patients with heart valve insufficiency (FIG. 4) and one without (FIG. 5) were examined with pulse acoustic analysis system, electrocardiograph and arterial blood pressure analyzer. The valve insufficiency could not be detected by either the electrocardiograph or arterial blood pressure analyzer, but could be detected by pulse acoustic analysis system. The plateau in the pulse acoustic graph indicated the heart valve insufficiency (FIG. 4). The pulse acoustic graph of the person without heart valve insufficiency did not show plateau but peaks (FIG. 5).

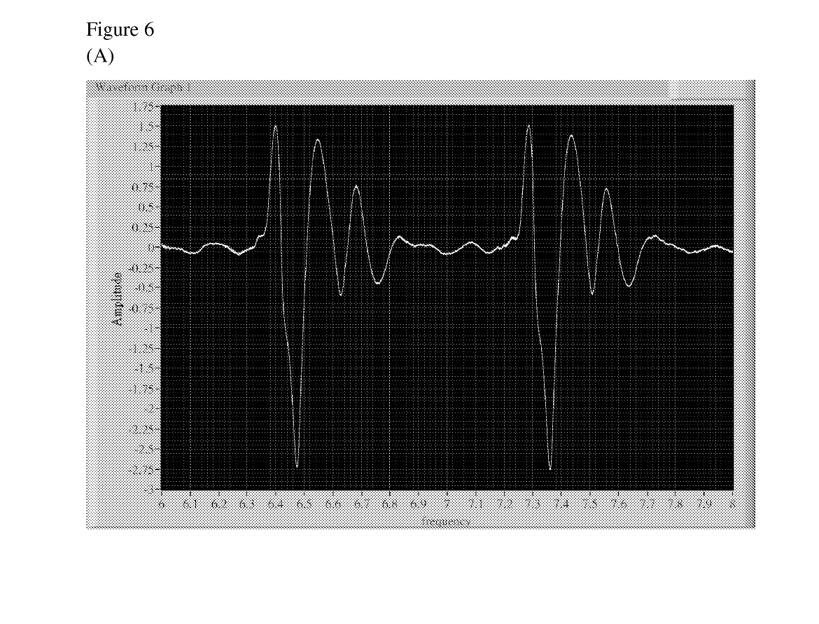

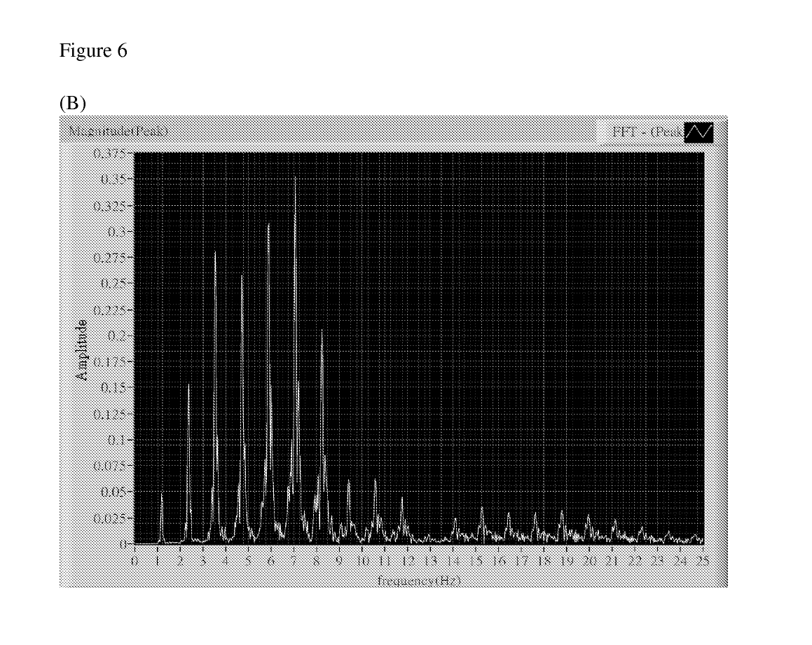

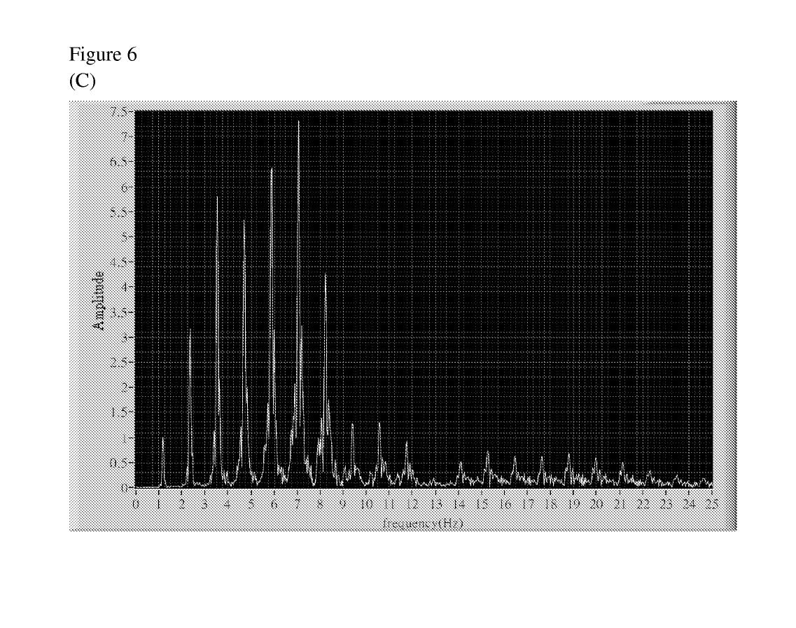

When people were examined with pulse acoustic analysis system, the data of the pulse acoustic waves indicated the blood flow to each organ according to the frequency of the waves, and the amplitude of the waves revealed blood supply information of organs in the subjects (FIG. 6).

While the invention has been described and exemplified in sufficient detail for those skilled in this art to make and use it, various alternatives, modifications, and improvements should be apparent without departing from the spirit and scope of the invention.

What is claimed is:

1. A pulse acoustic analysis system for detecting a cardiovascular disease or condition, comprising:
(a) a miniature acoustic sensor;
(b) a tool for fixing the miniature acoustic sensor;
(c) a signal amplifier connected to the miniature acoustic sensor;
(d) a digital-to-analog converter connected to signal amplifier; and
(e) a device, having a data collecting and processing program, which is connected to the digital-to-analog converter.

2. The system of claim 1, wherein the disease is a cardiovascular disease.

3. The system of claim 1, wherein the detection is directed to a dynamic state of blood output of a visceral organ.

4. The system of claim 3, wherein the visceral organ is a heart or a blood-vessel-related system.

5. The system of claim 1, wherein the device, having a data collecting and processing program, is a computer.

6. The system of claim 1, wherein the data collecting and processing program performs Fourier transformation.

7. The system of claim 1, wherein the tool is a wrist band which fixing the miniature acoustic sensor on a radial artery.

8. The system of claim 1 which further comprises an oscilloscope connected to the signal amplifier.

9. A method for detecting a dynamic pressure change in a blood dynamic system in a subject, comprising the following steps:
(a) fixing a pulse acoustic sensor, which consists of the miniature acoustic sensor and the tool of claim 1, on an artery to detect a pulse acoustic signal;
(b) amplifying the pulse acoustic signal by a signal amplifier;
(c) retrieving the signal by a digital-to-analog converter and storing the retrived signal in a computer;
(d) conducting a waveform analysis of the retrieved signals by a data collecting and processing program to generate a result of amplitude and frequency; and
(e) analyzing correlation between the result and the subject's health.

10. The method of claim 9, wherein the artery is radial artery, carotid artery or dorsalis pedis.

11. The method of claim 9, wherein the analysis comprises original waveform analysis, pulse acoustic waveform analysis and frequency spectrum analysis.

12. The method of claim 11, wherein the original waveform and the pulse acoustic waveform are displayed on a screen.

13. The method of claim 9, wherein the data collecting and processing program performs Fourier transformation.

14. The method of claim 11, wherein the original waveform analysis reveals a systole and a diastole of the heart and an on-off switch of cardiac valves.

15. The method of claim 11, wherein the frequency of the pulse acoustic waveform analysis reveals information of organs in the subject.

16. The method of claim 1, wherein the amplitude of the pulse acoustic waveform analysis reveals blood supply information of organs in the subject.

17. The method of claim 11, wherein the original waveform and the pulse acoustic waveform are output simultaneously, and compared, further with an electrocardiograph and an arterial blood pressure graph, to identify dynamic information of heart and blood vessel and dynamic state of blood output of branching blood vessels.

18. The system of claim 9, wherein the correlation between the result and the subject's health is further classified according to the type of cardiovascular disease.

19. The system of claim 9, wherein the subject is a human.
